(19) United States
(12) Patent Application Publication
(10) Pub. No.: US 2012/0171741 A1
(43) Pub. Date: Jul. 5, 2012

(54) Renewable Xylenes Produced from Bological C4 and C5 Molecules

(75) Inventors: Matthew W. Peters, Highlands Ranch, CO (US); Joshua D. Taylor, Evergreen, CO (US); Thomas Jackson Taylor, Highlands Ranch, CO (US); Leo E. Manzer, Wilmington, DE (US)

(73) Assignee: GEVO, Inc., Englewood, CO (US)

(21) Appl. No.: 13/286,741

(22) Filed: Nov. 1, 2011

Related U.S. Application Data
(60) Provisional application No. 61/409,092, filed on Nov. 1, 2010.

(51) Int. Cl. C12P 5/00

(52) U.S. Cl. 435/166

(57) ABSTRACT

The present invention is directed to a method for preparing renewable and relatively high purity p-xylene from biomass, and from C₅ molecules in particular. For example, biomass treated to provide a fermentation feedstock is fermented with a microorganism capable of producing a C₅ alcohol such as 3-methyl-1-butanol, followed by dehydration to provide a C₅ alkene such as 3-methyl-1-butanol, forming one or more C₈ olefins such as 2,5-dimethyl-3-hexene via metathesis, then dehydrocyclizing the C₈ olefins in the presence of a dehydrocyclization catalyst to selectively form renewable p-xylene with high overall yield.

### CROSS-REFERENCE TO RELATED APPLICATIONS

This application claims priority to U.S. provisional application Ser. No. 61/409,092 filed on Nov. 1, 2010, the entirety of which is incorporated herein by reference.

### BACKGROUND

Trimethylpentenes and trimethylpentanes can be produced by dimerization of isobutylene derived from renewable C₄ alcohols such as isobutanol. Conversion of trimethylpentenes and trimethylpentanes to p-xylene via known chemistry and/or convention routes typically limits the yield of xylenes (e.g., p-xylene) to less than 50% due to the tendency of these feed stocks to crack at the high temperatures required for these reactions. To avoid these yield losses, methods of converting isobutylene directly to 2,5-dimethylhexenes and 2,5-dimethylhexadienes, and subsequently cleanly converting the dienes to p-xylene at lower temperatures and high yields have been demonstrated, as disclosed in US Publication No. 2011/0087000 A1, for example. These methods generally require homogeneous metal catalysts such as alkyl aluminum salts, nickel phosphines, heterogeneous metal oxide catalysts, etc., often coupled with oxygen-mediated consumption of generated hydrogen—systems that are technically challenging to implement commercially. The present invention provides new and improved methods of converting renewable C₄ or C₅ molecules (e.g., butanols, butanals, pentanols, isoprene, etc.) to xylenes.

### SUMMARY OF THE INVENTION

The present invention is directed in various embodiments to methods for conversion of typical renewable C₄ and/or C₅ molecules (e.g., pentanols, isoprene, etc.) to renewable xylenes at high yield. In an embodiment, a process for preparing renewable p-xylene comprises treating biomass to form a feedstock and fermenting the feedstock with one or more species of microorganism, thereby forming one or more renewable C₄ or C₅ molecules, or a mixture thereof. The process also comprises reacting the renewable C₄ or C₅ molecules to form one or more renewable 2,5-dimethyl substituted-C₆ olefins, and dehydrogenating and aromatizing at least a portion of the one or more renewable 2,5-dimethyl substituted-C₆ olefins in the presence of a dehydrocyclization catalyst to form a mixture of comprising p-xylene and hydrogen. The process further comprises optionally isolating the renewable p-xylene.

### BRIEF DESCRIPTION OF THE DRAWINGS

FIG. 1 is an illustration of the generation of p-xylene from isobutanol in an exemplary embodiment of the invention;

FIG. 2 is an illustration of the generation of p-xylene from 3-methyl-1-butanol in an exemplary embodiment of the invention;

FIG. 3 is is an illustration of the generation of 2,5-dimethyl-3-hexene from 3-methyl-1-butanol in an exemplary embodiment of the invention;

FIG. 4 is an illustration of the generation of p-xylene from isoprene in an exemplary embodiment of the invention;

FIG. 5A is an illustration of the metathesis of isoprene to form a hexatriene mixture in an exemplary embodiment of the invention;

FIG. 5B is an illustration of the generation of p-xylene from the hexatriene mixture of FIG. 5A; and

FIG. 6 is an illustration of the generation of o-xylene from 2-methyl-1-butanol in an exemplary embodiment of the invention.

### DETAILED DESCRIPTION

All documents disclosed herein (including patents, journal references, etc.) are each incorporated by reference in their entirety for all purposes.

The term “microorganism” refers to single-celled organisms such as yeasts, fungi, bacteria (including cyanobacteria), eukaryotes, prokaryotes, algae, and archaea. Microorganisms convert a feedstock comprising carbon sources obtained from, for example biomass, to usable chemical products (e.g., one or more alcohols) which are converted using the methods of the present invention to produce isobutanol. The term “carbon source” generally refers to a substance suitable to be used as a source of carbon for prokaryotic or eukaryotic cell growth. Carbon sources include, but are not limited to biomass hydrolysates, starch, sucrose, cellulose, hemicellulose, xylose, and lignin, as well as monomeric components of these substrates. Carbon sources can comprise various organic compounds in various forms including, but not limited to, polymers, carbohydrates, acids, alcohols, aldehydes, ketones, amino acids, peptides, etc. These include, for example, various monosaccharides such as glucose, dextrose (D-glucose), maltose, oligosaccharides, polysaccharides, saturated or unsaturated fatty acids, succinate, lactate, acetate, ethanol, etc., or mixtures thereof. Photosynthetic organisms can additionally produce a carbon source directly from carbon dioxide as a product of photosynthesis. Photosynthetically derived carbon sources may be carbohydrates or intermediates and derivatives of intermediates found in carbohydrate-producing processes such as the Calvin cycle, gluconeogenesis, and the pentose phosphate pathway. For example, photosynthetically-produced pyruvate is a “carbon source” for cyanobacteria and algae. In some embodiments, carbon sources may be selected from biomass hydrolysates and glucose.

The term “biocatalyst” means a living system or cell of any type that speeds up chemical reactions by lowering the activation energy of the reaction and is neither consumed nor altered in the process. Biocatalysts may include, but are not limited to, microorganisms as described herein, such as yeasts, fungi, bacteria, and archaea.

The term “feedstock” is defined as a raw material or mixture of raw materials supplied to a microorganism or fermentation process from which other products can be made. For example, a carbon source such as biomass, the carbon compounds derived from biomass (e.g., a biomass hydrolysate as described herein), or traditional carbohydrates, are a feedstock for a microorganism that produces an alcohol or mixture of alcohols (e.g., ethanol and/or butanols) in a fermentation process. A feedstock may also contain nutrients other than the carbon source, needed by the microorganism to metabolize the feedstock. The term feedstock is used interchangeably with the term “renewable feedstock”, as the feedstocks used are generated from biomass or traditional carbohydrates, which are renewable substances.

When the microorganism is a photosynthetic organism such as algae or cyanobacteria, the term “feedstock” can include carbon dioxide and light, which the photosynthetic microorganism uses to form hydrocarbons, including carbohydrates and alcohols. Such microorganisms can utilize carbon dioxide, light, and carbohydrates into varieties of ways, for example under low light (or dark) conditions the microorganisms can ferment carbohydrates produced by photosynthesis or supplied to the microorganism, and under lighted conditions, the microorganisms can produce the desired alcohols from carbon dioxide and light directly. Furthermore, any of the microorganisms disclosed herein can be engineered to metabolize atypical feedstocks (e.g., other than biomass hydrolysate, traditional carbohydrates, etc.) such as carbon monoxide, acetate, glycerol, and petroleum-derived hydrocarbons to produce the desired alcohol product (e.g., isobutanol).

The term “traditional carbohydrates” refers to sugars and starches generated from specialized plants, such as sugar cane, sugar beets, corn, and wheat. Frequently, these specialized plants concentrate sugars and starches in portions of the plant, such as grains, that are harvested and processed to extract the sugars and starches. Traditional carbohydrates are often incorporated into food products derived from the nutrient rich protein component of these plants but generally offer little nutritional benefit to the animals that consume them. Industrial processing of traditional carbohydrates typically removes the starches and sugars from the nutrient dense portion of the plant (e.g. distillers grain from corn and gluten from wheat) and uses the carbohydrates as renewable feedstocks for fermentation processes which produce precursors to biofuels.

The term “biomass” as used herein refers primarily to the stems, leaves, and starch-containing portions of green plants, and is mainly comprised of starch, lignin, cellulose, hemicellulose, and/or pectin. Biomass can be decomposed by either chemical or enzymatic treatment to the monomeric sugars and phenols of which it is composed (Wyman, C. E. 2003 Biotechnological Progress 19:254-62). This resulting material, called biomass hydrolysate, is neutralized and treated to remove trace amounts of organic material that may adversely affect the microorganism(s), and is then used as a feedstock for fermentations. Alternatively, the biomass may be thermochemically treated to produce alcohols that may be further treated to produce biofuels and other valuable hydrocarbons.

The term “starch” as used herein refers to a polymer of glucose readily hydrolyzed by digestive enzymes. Starch is usually concentrated in specialized portions of plants, such as potatoes, corn kernels, rice grains, wheat grains, and sugar cane stems.

The term “lignin” as used herein refers to a polymer material, mainly composed of linked phenolic monomeric compounds, such as p-coumaryl alcohol, coniferyl alcohol, and sinapyl alcohol, which forms the basis of structural rigidity in plants and is frequently referred to as the woody portion of plants. Lignin is also considered to be the non-carbohydrate portion of the cell wall of plants.

The term “cellulose” as used herein refers is a long-chain polymer polysaccharide carbohydrate comprised of β-glucose monomer units, of formula (C₆H₁₀O₅)ₙ, usually found in plant cell walls in combination with lignin and any hemicellulose.

The term “hemicellulose” refers to a class of plant cell-wall polysaccharides that can be any of several heteropolymers. These include xylane, xyloglucan, arabinoxylan, arabinogalactan, glucuronoxylan, glucomannan and galactomannan. Monomeric components of hemicellulose include, but are not limited to: D-galactose, L-galactose, D-mannose, L-rhamnose, L-fucose, D-xylose, L-arabinose, and D-glucuronic acid. This class of polysaccharides is found in almost all cell walls along with cellulose. Hemicellulose is lower in weight than cellulose and cannot be extracted by hot water or chelating agents, but can be extracted by aqueous alkali. Polymeric chains of hemicellulose bind pectin and cellulose in a network of cross-linked fibers forming the cell walls of most plant cells.

The term “pectin” as used herein refers to a class of plant cell-wall heterogeneous polysaccharides that can be extracted by treatment with acids and chelating agents. Typically, 70-80% of pectin is found as a linear chain of α-(1-4)-linked D-galacturonic acid monomers. The smaller RG-I fraction of pectin is comprised of alternating (1-4)-linked galacturonic acid and (1-2)-linked L-rhamnose, with substantial arabinogalactan branching emanating from the rhamnose residue. Other monosaccharides, such as D-fucose, D-xylose, apiose, aceric acid, Kdo, Dha, 2-O-methyl-D-fucose, and 2-O-methyl-D-xylose, are found either in the RG-II pectin fraction (<2%), or as minor constituents in the RG-I fraction. Proportions of each of the monosaccharides in relation to D-galacturonic acid vary depending on the individual plant and its micro-environment, the species, and time during the growth cycle. For the same reasons, the homogalacturonan and RG-I fractions can differ widely in their content of methyl esters on GalA residues, and the content of acetyl residue esters on the C-2 and C-3 positions of GalA and neutral sugars.

The term “conversion” refers to the degree to which the reactants in a particular reaction (e.g., dehydration, metathesis, dehydrocyclization, etc.) are converted to products. Thus 100% conversion refers to complete consumption of reactants, and 0% conversion refers to no reaction.

The term “selectivity” refers to the degree to which a particular reaction forms a specific product, rather than another product. For example, for the dehydration of 3-methyl-1-butanol, 50% selectivity for 3-methyl-1-butene means that 50% of the alkene products formed are 3-methyl-1-butene, and 100% selectivity for 3-methyl-1-butene means that 100% of the alkene products formed are 3-methyl-1-butene. Because the selectivity is based on the product formed, selectivity is independent of the conversion or yield of the particular reaction.

The term “primarily” in reference to a component of a composition of the present invention (e.g., a composition comprised “primarily of 2-butene”) refers to a composition which comprises at least 50% of the referenced component.

The term “precursor” refers to an organic molecule in which all of the carbon contained within the molecule is derived from biomass, and is thermochemically or biochemically converted from a feedstock into the precursor.

The term “byproduct” means an undesired product related to the production of biofuel or biofuel precursor. Byproducts are generally disposed of as waste, thereby increasing the cost of the process.

The term “co-product” means a secondary or incidental product related to the production of biofuel. Co-products have potential commercial value that increases the overall value of biofuel production, and may be the deciding factor as to the viability of a particular biofuel production process.

The terms “alkene” and “olefin” are used interchangeably herein to refer to non-aromatic hydrocarbons having at least one carbon-carbon double bond. The term “diolefin” or “diene” then refers to a non-aromatic hydrocarbon having two carbon-carbon double bonds.

The term “aromatic compounds” or “aromatics” refers to hydrocarbons that contain at least one aromatic, six-membered ring. Non-limiting examples of aromatics relative to this invention are o-xylene, m-xylene, p-xylene, and other mono- and di-alkylated benzenes.

The term “dehydration” refers to a chemical reaction that converts an alcohol into its corresponding alkene. For example, the dehydration of isobutanol produces isobutene.

The term “oligomerization” or “oligomerizing” refer to processes in which molecules such as alkenes are combined with the assistance of a catalyst to form larger molecules called oligomers. Oligomerization refers to the combination of identical alkenes (e.g. ethene or isobutene) as well as the combination of different alkenes (e.g. ethyne and isobutene), or the combination of an unsaturated oligomer with an alkene. For example, butene (e.g., 1- and 2-butene) is oligomerized by an acidic catalyst to form eight-carbon compounds.

The term “aromatization” refers to processes in which hydrocarbon starting materials, typically alkenes or alkanes are converted into one or more aromatic compounds (e.g., p-xylene) in the presence of a suitable catalyst by dehydrocyclization.

“Dehydrocyclization” refers to a reaction in which an alkane or alkene is converted into an aromatic hydrocarbon and hydrogen, usually in the presence of a suitable dehydrocyclization catalyst, for example any of those described herein.

The phrase “substantially pure p-xylene” refers to isomeric composition of the xylenes produced by the dehydrocyclization step of the process. Xylenes which comprise “substantially pure p-xylene” comprise at least about 75% of the p-xylene isomer; and accordingly less than about 25% of the xylenes are other xylene isomers (e.g., o-xylene and m-xylene). Thus, xylenes comprising “substantially pure p-xylene” can comprise about 75%, about 80%, about 85%, about 90%, about 95%, about 96%, about 97%, about 98%, about 99%, about 99.5%, about 99.9%, or about 100% p-xylene.

“Renewably-based” or “renewable” denote that the carbon content of the precursor and subsequent products is from a “new carbon” source as measured by ASTM test method D 6866-05, “Determining the Biobased Content of Natural Range Materials Using Radiocarbon and Isotope Ratio Mass Spectrometry Analysis”, incorporated herein by reference in its entirety. This test method measures the ¹⁴C/¹²C isotope ratio in a sample and compares it to the ⁴C/¹²C isotope ratio in a standard 100% biobased material to give percent biobased content of the sample.

“Biobased materials” are organic materials in which the carbon comes from recently (on a human time scale) fixated CO₂ present in the atmosphere using sunlight energy (photosynthesis). On land, this CO₂ is captured or fixated by plant life (e.g., agricultural crops or forestry materials). In the oceans, the CO₂ is captured or fixated by photosynthesizing bacteria or phytoplankton. For example, a biobased material has a ¹⁴C/¹²C isotope ratio greater than 0. Contrarily, a fossil-based material, has a ¹⁴ C/¹² C isotope ratio of about 0. The term “renewable” with regard to compounds such as alcohols or hydrocarbons (linear or cyclic alkanes/alkenes/alkynes, aromatic, etc.) refers to compounds prepared from biomass using thermochemical methods (e.g., Fischer-Tropsch catalysts), biocatalysts (e.g., fermentation), or other processes, for example as described herein.

The term “rearrangement” refers to a chemical reaction in which alkyl groups on a hydrocarbon migrate to different positions on a carbon backbone molecule during a chemical reaction such as an oligomerization reaction. For example, the expected product of the dehydration of 1- or 2-butanol without rearrangement is 1- or 2-butene. With rearrangement, the migration of hydrogen and alkyl groups to other positions forms, for example, isobutene. Rearrangement can also refer to reactions in which the migration of a hydrogen atom changes the position of a carbon-carbon double bond in an alkene (for example, hydrogen migrations which interconvert 1-butene and 2-butenes).

The term “reaction zone” refers to the part of a reactor or series of reactors where the substrates and chemical intermediates contact a catalyst to ultimately form product. The reaction zone for a simple reaction may be a single vessel containing a single catalyst. For a reaction requiring two different catalysts, the reaction zone can be a single vessel containing a mixture of the two catalysts, a single vessel such as a tube reactor which contains the two catalysts in two separate layers, or two vessels with a separate catalyst in each which may be the same or different.

The term “saturated” refers to the oxidation state of a hydrocarbon molecule in which all bonds are single C—C or C—H bonds. Saturated acyclic hydrocarbons have a general molecular formula of CₙH₂ₙ₊₂.

“WHSV” refers to weight hourly space velocity, and equals the mass flow (units of mass/hr) divided by catalyst mass. For example, in a dehydration reactor with a 100 g dehydration catalyst bed, an isobutanol flow rate of 500 g/hr would provide a WHSV of 5 hr.sup.-1.

Unless otherwise indicated, all percentages herein are by weight (i.e., “wt. %).

Various embodiments of the present invention are directed to methods for converting renewable C₄ and C₅ molecules into unsaturated C8 hydrocarbons that may subsequently be converted into single xylene isomers. Non-limiting examples of C₄ molecules include butanols such as 1-butanol, 2-butanol, t-butanol, isobutanol, butyraldehyde, and isobutyraldehyde, etc. Non-limiting examples of C₅ molecules include, for example, 3-methyl-1-butanol, 2-methyl-1-butanol, 3-methyl-1-butene, 3-methyl-2-butene, and isoprene. Non-limiting examples of unsaturated C₈ hydrocarbons (e.g., dimethyl substituted C₆ olefins) include 2,5-dimethyl-3-hexene, 2,5-dimethyl-2,4-hexadiene, 2,5-dimethyl-1,5-hexadiene, 2,5-dimethyl-1,3,5-hexatriene, 3,4-dimethyl-1,3,5-hexatriene, and 2,4-dimethyl-1,3,5-hexatriene, etc. The single xylene isomers comprise o-xylene, p-xylene, m-xylene, or mixtures thereof. The renewable C₄ and C₅ precursor molecules may be produced by microorganisms naturally or through genetic modification of metabolic pathways to overproduce these compounds.

In most embodiments, the C₄ or C₅ molecules are obtained from fermentation of a suitable feedstock. The fermentation feedstock typically comprises a carbon source obtained from treating biomass. Suitable carbon sources include any of those described in U.S. Pub. No. 2011/0087000, such as starch, mono- and polysaccharides, pre-treated cellulose and hemicellulose, lignin, pectin, etc., which are obtained by subjecting biomass to one or more processes known in the art, including extraction, acid hydrolysis, enzymatic treatment, etc.

The C₄ or C₅ molecules produced during fermentation can be removed from the fermentation broth by various methods, for example fractional distillation, solvent extraction (e.g., in particular embodiments with a renewable solvent such as renewable oligomerized hydrocarbons, renewable hydrogenated hydrocarbons, renewable aromatic hydrocarbons, etc. prepared as described herein), adsorption, pervaporation, etc. or by combinations of such methods, prior to dehydration. In other embodiments, the alcohol produced during fermentation is not isolated from the fermentation broth prior to dehydration, but is dehydrated directly as a dilute aqueous solution. The removal of C₄ or C₅ molecules from the fermentation broth, as described herein, can occur continuously or semi-continuously. Removal of the C₄ or C₅ molecules is advantageous because it provides for separation of the C₄ or C₅ molecules from the fermentation broth and removes a metabolic by-product of the fermentation, thereby improving the productivity of the fermentation process.

The carbon source is converted into a precursor of xylenes (e.g., isobutanol, 2-methyl-1-butanol, 3-methyl-1-butanol, 2-methyl-1-butene, 3-methyl-1-butene, isoprene, etc.) by the metabolic action of a biocatalyst (or by thermochemical methods, e.g. using gasification followed by chemical reaction over Fischer-Tropsch catalysts). The carbon source is consumed by the biocatalyst (e.g., a living system or cell of any type that speeds up chemical reactions by lowering the activation energy of the reaction and is neither consumed nor altered in the process). Biocatalysts may include, but are not limited to, microorganisms such as yeasts, fungi, bacteria, and archaea. The carbon source is then excreted as a xylene precursor, for example as a C₄ and/or C₅ molecule, (e.g., isobutanol, 2-methyl-1-butanol, 3-methyl-1-butanol, 2-methyl-1-butene, 3-methyl-1-butene, isoprene, etc.) in a large fermentation vessel. The xylene precursor is then separated from the fermentation broth, optionally purified, and then subjected to further processes such as dehydration, dehydrogenation, dimerization, metathesis, etc. to form suitable C₈ hydrocarbons (e.g., dimethyl substituted C₆ olefins), which are then aromatized and/or dehydrocyclized to form aromatics comprising xylenes, either as a mixture of isomers or as substantially enriched in one isomer (e.g., p-xylene). When the carbon source is renewable, the formed aromatics are renewable.

Each of the various reaction steps (e.g. dehydration, dehydrogenation, dimerization, metathesis, aromatization, and dehydrocyclization) are preferably carried out under reaction conditions which favor selectively forming specific products. For example, the dehydrocyclization reaction is carried out in the presence of a particular dehydrocyclization catalyst, and under particular temperature, pressure, diluent and WHSV conditions which selectively form p-xylene (e.g., at least about 75% of the xylenes formed are p-xylene) or any other desirable xylene isomer/composition.

Selective dehydration, dehydrogenation, dimerization, metathesis, aromatization, and dehydrocyclization reaction steps are promoted by a variety of methods which reduce unwanted side-reactions (and the resulting undesirable by-products), such as the use of particularly selective catalysts, the addition of diluents, reduced reaction temperatures, reduced reactant residence time over the catalyst (i.e., higher WHSV values), etc. Such reaction conditions tend to reduce the percent conversion of particular reaction steps below 100%, and thus the feedstock for each successive reaction can include unreacted starting materials from the previous reaction step (which can function as diluents, as well as added diluents and by-products from previous reaction steps; For example, the feedstock for the dehydrocyclization reaction step can include the C₈ olefin produced by a metathesis reaction, as well as diluent gases (e.g., nitrogen, argon, and methane), unreacted C₅ alkene, etc. from the metathesis reaction, other by-product C₅ and/or C₈ molecules from the dehydrocyclization reaction, etc. Unreacted starting materials can also be recycled back to the appropriate reaction step in order to boost the overall yield of p-xylene. For example, unreacted C₅ alkene present in the product stream from the metathesis reaction (or in some cases, also present in the product stream from the dehydrocyclization reaction) can be separated out of the product stream and recycled back to the feedstock for the metathesis reaction. In addition, C₅ and C₈ by-products formed during the dehydrocyclization reaction (e.g., from the corresponding C₅ and C₈ alkenes present in the dehydrocyclization feedstock) can be recycled back to the feedstock for the dehydrocyclization reaction.

The various reaction steps subsequent to production of the C₄ and/or C₅ molecules (such as dehydration, metathesis, dehydrocyclization, etc.) can be carried out in a single reactor, within which the individual reaction steps take place in different reaction zones; or in which the catalysts are mixed or layered together in a single reaction zone, whereby the C₄ and/or C5 molecules undergoes sequential conversion to successive intermediates in a single reaction zone (e.g., conversion of a C₅ alcohol to a C₅ alkene, then a C₈ alkene in a single reaction zone; or conversion of a C₅ alkene to a C₈ alkene, then dehydrocyclization of the C₈ alkene to p-xylene in a single reaction zone).

Alternatively, the various reactions can be carried out in separate reactors so that the reactor conditions (e.g., temperature, pressure, catalyst, feedstock composition, WHSV, etc.) can be optimized to maximize the selectivity of each reaction step. When the separate reaction steps are carried out in separate reactors, the intermediates formed in the various reaction steps can be isolated and/or purified before proceeding to the subsequent reaction step, or the reaction product from one reactor can be passed directly to the subsequent reactor without purification.

In other embodiments of the processes of the present invention, one or more of the particular reaction steps (such as dehydration, metathesis, dehydrocyclization, etc.) can each be carried out in two or more reactors (connected either in series or in parallel), so that during operation of the process, particular reactors can be bypassed (or taken “offline”) to allow maintenance (e.g., catalyst regeneration) to be carried out on the bypassed reactor, while still permitting the process to continue in the remaining operational reactors. For example, the dehydrocyclization step could be carried out in two reactors connected in series (whereby the product of the metathesis step is the feedstock for the first dehydrocyclization reactor, and the product of the first dehydrocyclization reactor is the feedstock for the second dehydrocyclization reactor). The first dehydrocyclization reactor can be bypassed using the appropriate piping and valves such that the product of the dehydrocyclization step is now the feedstock for the second dehydrocyclization reactor. For reactors connected in parallel, bypassing one of the reactors may simply entail closing the feed and product lines of the desired reactor. Such reactor configurations, and means for by-passing or isolating one or more reactors connected in series or parallel are known in the art.

Depending on the biocatalyst employed, a particular C₄ and/or C₅ molecule or a mixture of C₄ and/or C₅ molecules can be obtained. For example, the biocatalyst can be a single microorganism capable of forming more than one type of C₄ and/or C₅ alcohol(s) during fermentation (e.g. two or more of 1-butanol, isobutanol, 2-butanol, t-butanol, 3-methyl-1-butanol, 2-methyl-1-butanol, etc.). In most embodiments however, it is generally advantageous to obtain primarily one type of C₄ or C₅ alcohol. In one embodiment, the C₄ alcohol is isobutanol. In another embodiment, the C₅ alcohol is 3-methyl-1-butanol or 2-methyl-1-butanol. In most embodiments, a particular microorganism which preferentially forms C₄ and/or C₅ molecules, for example alcohols, during fermentation is used.

In addition or alternatively, renewable C₄ and/or C₅ alcohols may be prepared photosynthetically using an appropriate photosynthetic organism. Renewable alcohols can be prepared photosynthetically, e.g., using cyanobacteria or algae engineered to produce isobutanol, isopentanol, and/or other alcohols (e.g., Synechococcus elongatus PCC7942 and Synechocystis PCC6803; see Angermayr et al., Energy Biotechnology with Cyanobacteria, Current Opinion in Biotechnology 2009, 20, 257-263, Atsumi and Liao, Nature Biotechnology, 2009, 27, 1177-1182); and Dexter et al., Energy Environ. Sci., 2009, 2, 857-864, and references cited in each of these references). When produced photosynthetically, the “feedstock” for producing the resulting renewable alcohols is the light and the CO₂ provided to the photosynthetic organism (e.g., cyanobacteria or algae).

Any suitable organism which produces a C₄ and/or C₅ alcohol can be used in a fermentation step to provide the xylene precursor(s) described herein. For example, alcohols such as isobutanol are produced by yeasts during the fermentation of sugars into ethanol. Such alcohols (termed fusel alcohols in the art of industrial fermentations for the production of beer and wine) have been studied extensively for their effect on the taste and stability of these products. Recently, production of fusel alcohols using engineered microorganisms has been reported (see, e.g., U.S. Patent Publication No. 2007/0092957, and Nature, 2008, 451, p. 86-89). Isobutanol can be fermentatively produced by recombinant microorganisms as described in U.S. Provisional Patent Application No. 60/730,290 or in U.S. Patent Publ. Nos. 2009/0226990, 2009/0226991, 2009/0215137, 2009/0171129; 2-butanol can be fermentatively produced by recombinant microorganisms as described in U.S. Patent Application No. 60/796,816; and 1-butanol can be fermentatively produced by recombinant microorganisms as described in U.S. Provisional Patent Application No. 60/721,677. Other suitable microorganisms may include those described, for example in U.S. Patent Publ. Nos. 2008/0293125, 2009/0155869.

In addition or alternatively, embodiments of the present invention may employ a C₅ diene (e.g., isoprene) as a xylene precursor. Isoprene may also be produced by biocatalysts as described in, e.g., U.S. patent application Ser. No. 12/659,216, the relevant portions of which are incorporated herein by reference. Suitable biocatalysts include any microbial host cells capable of making isoprene, for example microbes such as those described by U.S. Pat. No. 5,849,970 (herein incorporated by reference) and including Bacillus amyloliquiefaciens; Bacillus cereus; Bacillus subtillis 6051; Basillus substillis 23059; Bacillus subtillis 23856; Micrococcus luteus; Rhococcus rhodochrous; Acinetobacter calcoacetiucus; Agrobacternum rhizogenes; Escherichia coli; Erwinia herbicola; Pseudomonoas aeruginosa; and Pseudomonas citronellolis. Since microbes which naturally produce isoprene do so at low levels, such microbes can be modified, for example by the insertion of isoprene synthase into its genome. Illustrative examples of suitable nucleotide sequences include but are not limited to: (EF638224, Populus alba); (AJ294819, Populus alba.times.Populus tremula); (AM410988, Populus nigra); (AY341431, Populus tremuloides); (EF147555, Populus trichocarpa); and (AY316691, Pueraria montana var. lobata). The addition of a heterologous isoprene synthase to a microbial host cells that make isoprene naturally will improve isoprene yields of natural isoprene producers as well. Any suitable microbial host cell can be genetically modified to make isoprene. A genetically modified host cell is one in which nucleic acid molecules have been inserted, deleted or modified (i.e., mutated; e.g., by insertion, deletion, substitution, and/or inversion of nucleotides), to produce isoprene. Illustrative examples of suitable host cells include any archae, bacterial, or eukaryotic cell. Examples of archae cells include, but are not limited to those belonging to the genera: Aeropyrum, Archaeglobus, Halobacterium, Methanococcus, Methanobacterium, Pyrococcus, Sulfolobus, and Thermoplasma. Illustrative examples of archae species include but are not limited to: Aeropyrum pernix, Archaeoglobus fulgidus, Methanococcus jannaschii, Methanobacterium thermoautotrophicum, Pyrococcus abyssi, Pyrococcus horikoshii, Thermoplasma acidophilum, Thermoplasma volcanium. Examples of bacterial cells include, but are not limited to those belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphlococcus, Strepromyces, Synnecoccus, and Zymomonas. Illustrative examples of bacterial species include but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, Staphylococcus aureus, and the like. In general, if a bacterial host cell is used, a non-pathogenic strain is preferred. Illustrative examples of species with non-pathogenic strains include but are not limited to: Bacillus subtilis, Escherichia coli, Lactibacillus acidophilus, Lactobacillus helveticus, Pseudomonas aeruginosa, Pseudomonas mevalonii, Pseudomonas pudita, Rhodobacter sphaeroides, Rodobacter capsulatus, Rhodospirillum rubrum, and the like. Examples of eukaryotic cells include but are not limited to fungal cells. Examples of fungal cells include, but are not limited to those belonging to the genera: Aspergillus, Candida, Chrysosporium, Cryotococcus, Fusarium, Kluyveromyces, Neotyphodium, Neurospora, Penicillium, Pichia, Saccharomyces, Trichoderma and Xanthophyllomyces (formerly Phaffia). Illustrative examples of eukaryotic species include but are not limited to: Aspergillus nidulans, Aspergillus niger, Aspergillus oryzae, Candida albicans, Chrysosporium lucknowense, Fusarium graminearum, Fusarium venenatum, Kluyveromyces lactis, Neurospora crassa, Pichia angusta, Pichia finlandica, Pichia kodamae, Pichia membranaefaciens, Pichia methanolica, Pichia opuntiae, Pichia pastoris, Pichia pijperi, Pichia quercuum, Pichia salictaria, Pichia thermotolerans, Pichia trehalophila, Pichia stipitis, Streptomyces ambofaciens, Streptomyces aureofaciens, Streptomyces aureus, Saccaromyces bayanus, Saccaromyces boulardi, Saccharomyces cerevisiae, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus, Trichoderma reesei and Xanthophyllomyces dendrorhous (formerly Phaffia rhodozyma).

In general, if a eukaryotic cell is used, a non-pathogenic strain is preferred. Illustrative examples of species with non-pathogenic strains include but are not limited to: Fusarium graminearum, Fusarium venenatum, Pichia pastoris, Saccaromyces boulardi, and Saccaromyces cerevisiae. In some embodiments, the host cells of the present invention have been designated by the Food and Drug Administration as GRAS or Generally Regarded As Safe. Illustrative examples of such strains include: Bacillus subtilis, Lactibacillus acidophilus, Lactobacillus helveticus, and Saccharomyces cerevisiae. In addition to the heterologous nucleic acid encoding an isoprene synthase, the microbial host cell can be further modified to increase isoprene yields. These modifications include but are not limited to the expression of one or more heterologous nucleic acid molecules encoding one or more enzymes in the mevalonate or DXP pathways. Isoprene may also be produced directly from carbon dioxide and light in natural or engineered photosynthetic organisms as described above.

Alternatively, butadiene (or isoprene) may be produced via sequential dehydration and dehydrogenation reactions from a butanol (or pentanol) feedstock, for example isobutanol (or 3-methyl-1-butanol). For example, dehydration of butanol (or 3-methyl-1-butanol) provides a relatively simple mixture of butene (or methylbutene) isomers which can be converted directly to butadiene (or isoprene) by dehydrogenation. Any byproduct of the dehydration which cannot be converted directly to butadiene (or isoprene) can be readily removed, either from the mixture of linear butene isomers (or methylbutene) isomers, or from the butadiene (or isoprene) of the product stream of the dehydrogenation step. See, e.g., U.S. Pub. No. 2010/0216958, which is incorporated herein by reference in its entirety. In addition or alternatively, renewable isoprene may be produced from renewable isobutanol by dehydration of the renewable isobutanol to isobutylene, followed by condensation with formaldehyde, as, for example, in a Prins reaction.

Embodiments of the present invention provide novel routes to selectively convert biological C₄ and C₅ molecules to specific xylene isomers, especially p-xylene. Certain embodiments will be described in further detail below as examples and with reference to the appended figures. The following exemplary embodiments should be understood to be illustrative of the present invention, and should not be construed as limiting or non-overlapping. On the contrary, the present disclosure embraces alternatives and equivalents thereof. All documents disclosed herein (including patents, journal references, ASTM methods, etc.) are each incorporated by reference in their entirety for all purposes.

### EXAMPLE 1

### Xylenes Via Oxidation of C₄ Alcohols to C₄ Aldehydes.

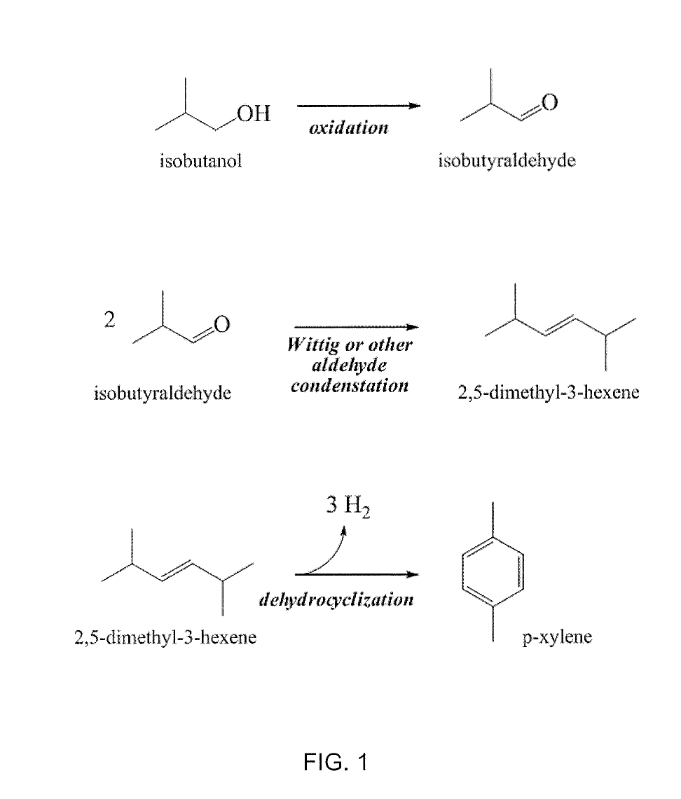

In general, a renewable C₄ alcohol (e.g., isobutanol) may be oxidized to produce a corresponding C₄ aldehyde (e.g., isobutyraldehyde). Selective oxidation of alcohols to aldehydes may be affected by employing transition metal oxidants (Cr, Fe or Mn based reagents, etc), by employing sulfur-based oxidants (e.g., Swern-type reagents), or by employing hypervalent iodine reagents (e.g., Dess-Martin periodinane, etc.). Subsequent homocoupling of a resultant aldehyde by one or more processes such as aldol-type coupling (and optionally subsequent dehydration and/or dehydrogenation) may afford the desired C₈ olefin (e.g., 2,5,-dimethyl-3-hexene) as exemplified in FIG. 1.

In addition or alternatively, other suitable olefin-generating chemistry (e.g., Wittig-type coupling) can yield a desired product C₈ olefin. As shown in FIG. 1, the Wittig reaction of isobutyraldehyde with a suitable coupling partner such as an isobutyl halide (e.g., isobutyl bromide) yields the desired 2,5-dimethyl-3-hexene. The target 2,5-dimethyl-3-hexene may optionally be separated or purified, then selectively converted to p-xylene (or a mixture of p-xylene and other isomers).

Conversion to p-xylene (or a mixture of p-xylene and other isomers) may be effected by dehydrocyclization of 2,5-dimethyl-3-hexene. For example, as shown in FIG. 1, 2,5-dimethyl-3-hexene may be subject to dehydrogenation and aromatization (“dehydrocyclization”) conditions to afford p-xylene. Beneficially, the dehydrogenation and aromatization is performed in a single reaction zone and over a single dehydrocyclization catalyst.

P-xylene (and other aromatics) are currently produced by catalytic cracking and catalytic reforming of petroleum-derived feedstocks. In particular, the catalytic reforming process uses light hydrocarbon “cuts” like liquefied petroleum gas (C₃ and C₄) or light naphtha (especially C₅ and C₆), which are then converted to C₆-C₈ aromatics, typically by one of the three main petrochemical processes such as M-2 Forming (Mobil), Cyclar (UOP), and Aroforming (IFP-Salutec). These petrochemical processes use new catalysts which were developed to produce petrochemical grade benzene, toluene, and xylene (BTX) from low molecular weight alkanes in a single step. The process can be described as dehydrogenation and dehydrocyclooligomerization over one catalyst and in a single reaction zone (the use of C₃ hydrocarbons requires oligomerization rather than dimerization to prepare substituted aromatics).

A variety of alumina and silica based dehydrocyclization catalysts and reactor configurations may be used in the present invention to prepare aromatics such as p-xylene from low molecular weight hydrocarbons, such as C₄ and C₅ molecules. For example, the Cyclar process for converting liquefied petroleum gas into aromatic compounds uses a gallium-doped zeolite (Appl. Catal. A, 1992, 89, p. 1-30). Other catalysts include bismuth, lead, or antimony oxides (U.S. Pat. No. 3,644,550 and U.S. Pat. No. 3,830,866), chromium treated alumina (U.S. Pat. No. 3,836,603 and U.S. Pat. No. 6,600,081), rhenium treated alumina (U.S. Pat. No. 4,229,320) and platinum treated zeolites (WO 2005/065393 A2). A non-limiting list of such dehydrocyclization catalysts include mixtures of chromia-alumina and bismuth oxide (e.g., bismuth oxide prepared by the thermal decomposition of bismuth compounds such as bismuth nitrate, bismuth carbonate, bismuth hydroxide, bismuth acetate, etc. and e.g., chromia-alumina prepared by impregnating alumina particles with a chromium composition to provide particles containing about 5, about 10, about 15, about 20, about 25, about 30, about 35, about 40, about 45, or about 50 mol % chromia, optionally including a promoter such as potassium, sodium, or silicon, and optionally including a diluent such as silicon carbide, α-alumina, zirconium oxide, etc.); bismuth oxide, lead oxide or antimony oxide in combination with supported platinum, supported palladium, supported cobalt, or a metal oxide or mixtures thereof, such as chromia-alumina, cobalt molybdate, tin oxide or zinc oxide; supported chromium on a refractory inorganic oxide such as alumina or zirconia, promoted with metal such as iron, tin, tungsten, optionally in combination with a Group I or II metal such as Na, K, Rb, Cs, Mg, Ca, Sr, and Ba); rhenium in oxide or metallic form deposited on a neutral or weakly acidic support which has been additionally impregnated with an alkali metal hydroxide or stannate and subsequently reduced with hydrogen at elevated temperatures; and platinum deposited on aluminosilicate MFI zeolite.

Any of these known catalysts can be used in the process of the present invention to form the desired xylene product. Where a mixture or distribution of products is obtained, such a mixture may be separated by conventional techniques known in the art (e.g., distillation, etc.) to afford p-xylene (or other reaction products) at a desired purity level (e.g., >90%, >95%, >99%, etc.). The ethylene obtained from the metathesis reaction may be converted to renewable polyethylene glycol or ethylene glycol.

High selectivity for p-xylene in the dehydrocyclization reaction is favored by appropriate selection of dehydrocyclization catalyst (as described above), and by appropriate selection of dehydrocyclization process conditions (e.g., process temperature, pressure, WHSV, etc.). In most embodiments, the dehydrocyclization reaction is carried out below or slightly above atmospheric pressure, for example at pressures ranging from about 1 psia to about 20 psia, or about 1 psia, about 2 psia, about 3 psia, about 4 psia, about 5 psia, about 6 psia, about 7 psia, about 8 psia, about 9 psia, about 10 psia, about 11 psia, about 12 psia, about 13 psia, about 14 psia, about 15 psia, about 16 psia, about 17 psia, about 18 psia, about 19 psia, and about 20 psia, inclusive of all ranges and subranges therebetween. In most embodiments, the dehydrocyclization is carried out at temperatures ranging from about 300° C. to about 600° C., or about 300° C., about 325° C., about 350° C., about 375° C., about 400° C., about 425° C., about 450° C., about 475° C., about 500° C., about 525° C., about 550° C., about 575° C., and about 600° C., inclusive of all ranges and subranges therebetween. In most embodiments, the dehydrocyclization is carried out at WHSV values of about 1 hr⁻¹, for example about 0.51 h⁻¹, about 1 hr⁻¹, about 1.5 hr⁻¹, or about 2 hr⁻¹, inclusive of all ranges and subranges therebetween. In most embodiments, the dehydrocyclization reaction is operated at conversions ranging from about 40-95%, and provides a p-xylene selectivity (i.e., the percentage of xylene products which is p-xylene) greater than about 75%. In other embodiments, the p-xylene selectivity is ≧ about 75%, ≧ about 80%, ≧ about 85%, ≧ about 90%, ≧ about 95%, ≧ about 96%, ≧ about 97%, ≧ about 98%, or ≧ about 99%.

In addition, both the conversion and selectivity of the dehydrocyclization reaction for p-xylene can be enhanced by adding diluents to the feedstock, such as hydrogen, nitrogen, argon, and methane. Unreacted C₅ alkene, for example, can also be used as an effective diluent to improve the p-xylene selectivity of the dehydrocyclization reaction, and to help suppress cracking.

The dehydrocyclization reaction step of the present invention is typically carried out in the relative absence of oxygen (although trace levels of oxygen may be present due to leaks in the reactor system, and/or the feedstock for the dehydrocyclization reaction step may have trace contamination with oxygen). The hydrogen and light hydrocarbons produced as a by-product of the dehydrocyclization reaction are themselves valuable compounds that can be removed and used for other chemical processes (e.g., hydrogenation of alkene by-products, for example C₈ alkenes such as 2,4,4-trimethylpentenes) to produce alkanes suitable for use as renewable fuels or renewable fuel additives (e.g., isooctane), etc.). In analogy to the practice in traditional petrochemical refineries that produces aromatics, these light compounds can be collected and used.

Typically, the high temperatures at which these dehydrocyclization reactions are carried out tend to coke up and deactivate the dehydrocyclization catalysts. To reuse the dehydrocyclization catalyst, the coke must be removed as frequently as every 15 minutes, usually by burning it off in the presence of air. Thus, even though the dehydrocyclization reaction itself is, in most embodiments of the present invention, carried out in the absence of oxygen, oxygen (and optionally hydrogen) can periodically be introduced to reactivate the dehydrocyclization catalyst. The presence of hydrogenating metals such as nickel, platinum, and palladium in the catalyst will catalyze the hydrogenation of the coke deposits and extend dehydrocyclization catalyst life. In order to accommodate reactivation of the catalyst in a continuous process, two or more dehydrocyclization reactors can be used so that at least one dehydrocyclization reactor is operational while other dehydrocyclization reactors are taken “off line” in order to reactivate the catalyst. When multiple dehydrocyclization reactors are used, they can be connected in parallel or in series.

As discussed above, the hydrocarbon feedstocks used to form aromatic compounds in conventional petroleum refineries are typically mixtures of hydrocarbons. As a result, the p-xylene produced by petroleum refineries is mixed with other xylene isomers and other aromatics (e.g., light aromatics such as benzene and toluene, as well as ethylbenzene, etc.), requiring further separation and purification steps in order to provide suitably pure p-xylene, which may be required for subsequent conversion, to terephthalic acid or terephthalate esters suitable for polyester production, for example. In a large-scale refinery, producing pure streams of p-xylene can be expensive and difficult. In contrast, the process of the present invention can readily provide relatively pure, renewable p-xylene at a cost which is competitive with that of petroleum derived p-xylene from conventional refineries.

### EXAMPLE 2

### Xylenes Via Dehydration of C₅ Alcohols to C₅ Alkenes.

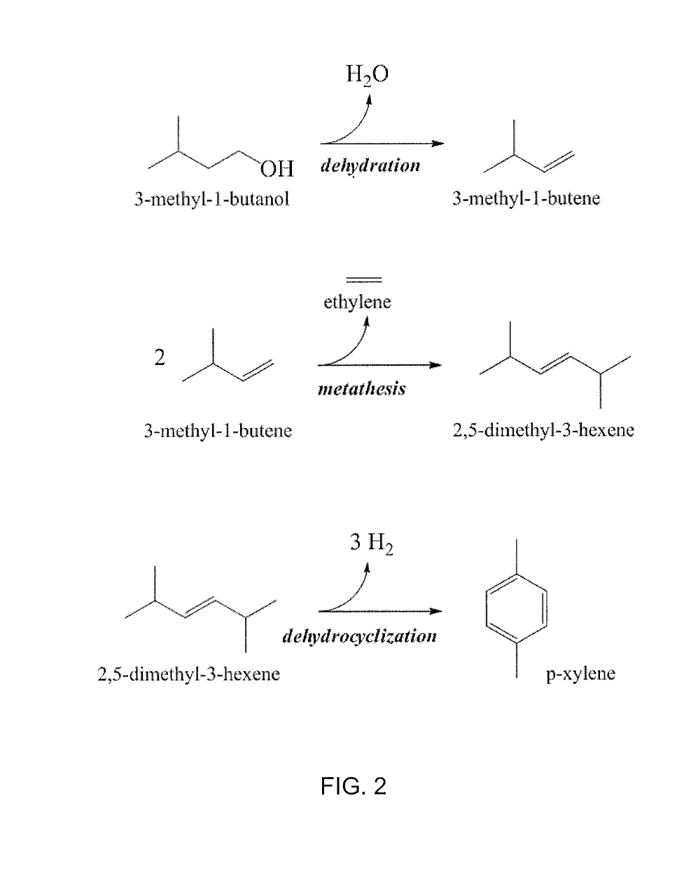

As shown in FIG. 2, in general, a C₅ alcohol (e.g. 3-methyl-1-butanol) may be dehydrated to form a corresponding C₅ alkene (e.g. 3-methyl-1-butene). Dehydration may be effected by techniques as described in, e.g., U.S. patent application Ser. No. 12/899,285. The product resulting 3-methyl-1-butene may then be subsequently subject to homometathesis with a metathesis catalyst under conditions that favor the removal of ethylene to form 2,5-dimethyl-3-hexene. Any suitable catalyst for promoting olefin metathesis may be employed (e.g., a Ru-based catalyst or other any suitable transition metal catalyst known in the art). As shown in FIG. 2, homometathesis of 3-methyl-1-butene affords 2,5-dimethyl-3-hexene and ethylene, which may be removed from the reaction space during the metathesis reaction. The 2,5-dimethyl-3-hexene product may optionally be separated or purified, and then selectively converted to p-xylene via dehydrocyclization as previously described in Example 1 to afford p-xylene or a mixture of isomeric xylenes. Separation of a product mixture comprising p-xylene, other isomeric xylenes, unreacted olefins and/or side products may be performed to afford p-xylene (or other reaction products) at a desired purity level (e.g., >90%, >95%, >99%, etc.).

### EXAMPLE 3

### Dehydration of C₅ Alcohols to Form a Mixture of Isomeric C₅ Alkenes.

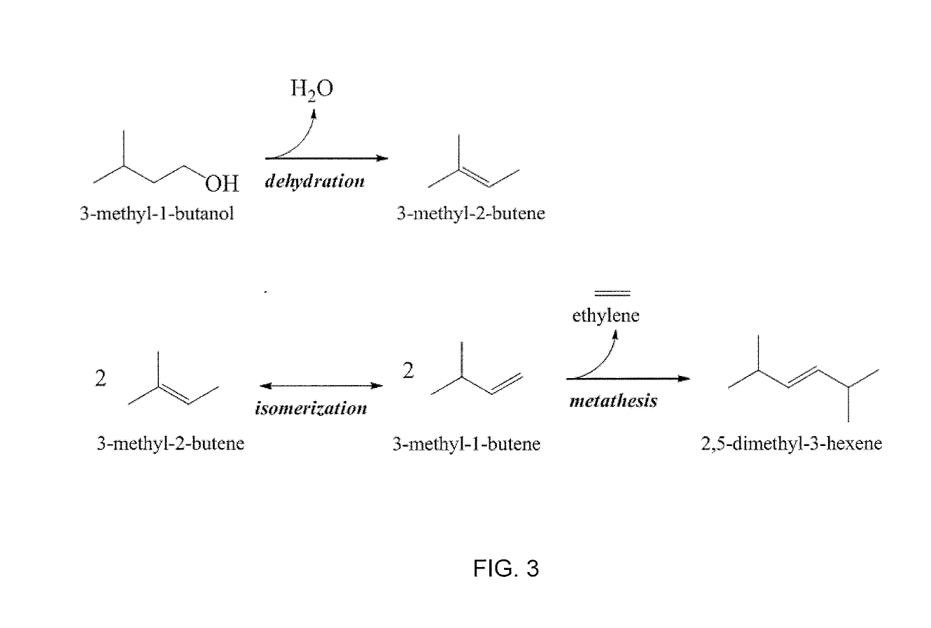

As shown in FIG. 3, a mixture of isomeric butenes may be formed via dehydration of a corresponding alcohol (e.g., 3-methyl-1-butanol) as previously described herein. The product butenes (e.g., 3-methyl-2-butene and 3-methyl-1-butene) may be treated with an isomerization catalyst to provide a desired butene isomer, or to enrich a mixture of butenes in a desired isomer. Suitable isomerization catalysts are any catalyst known in the art for promoting isomerization of olefins, including but not limited to acidic catalysts, and metal catalysts such as MgO. A product butene (or mixture of butenes) may then be subject to metathesis conditions (e.g., employing an olefin metathesis catalyst as previously described herein), affording ethylene and 2,5-dimethyl-3-hexene (or isomers thereof). As shown in FIG. 3, product 2,5-dimethyl-3-hexene is formed via homometathesis of 3-methyl-1-butene. The product 2,5-dimethyl-3-hexene may then be selectively converted to p-xylene via dehydrocyclization as previously described in Example 1. Separation of a product mixture comprising p-xylene, other isomeric xylenes, unreacted starting olefin and/or side products may be performed to afford p-xylene (or other reaction products) at a desired purity level (e.g., >90%, >95%, >99%, etc.).

### EXAMPLE 4

### Formation of Xylene Via Metathesis of C₅ Alkenes Over Inactive Metathesis Catalyst.

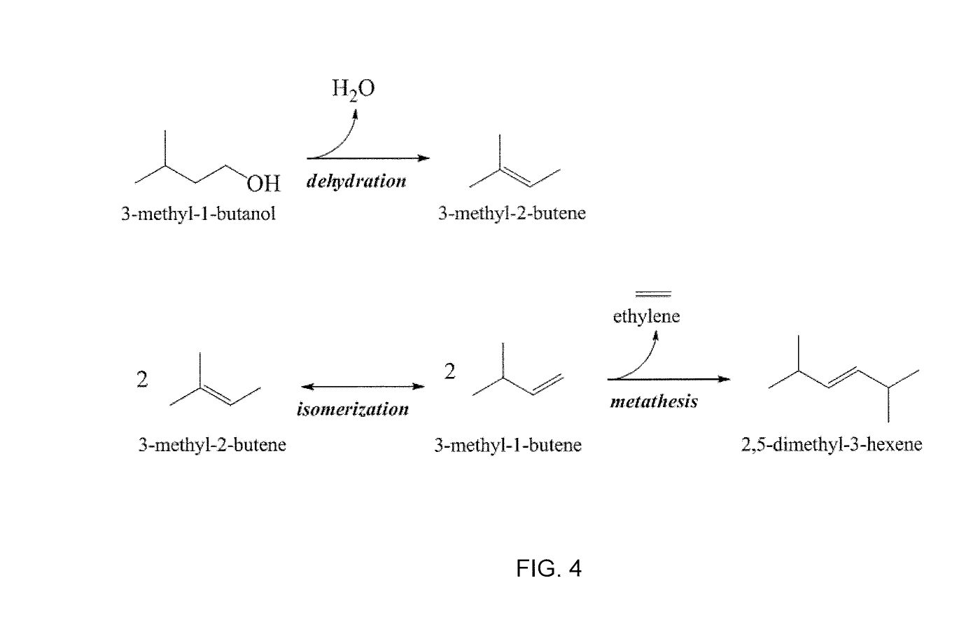

As shown in FIG. 4, isoprene (an exemplary C₅ alkene) may also be employed in the formation of xylenes (e.g., p-xylene). As previously described herein, isoprene may be renewably obtained by any suitable means, e.g., via biocatalysis directly, and/or by dehydration and dehydrogenation of, e.g., renewable isobutanol. For example, isoprene may also be obtained by dehydration of renewable pentanol to renewable pentene, followed by dehydrogenation of the renewable pentene to renewable isoprene.

Subsequent homometathesis of isoprene over an inactive metathesis catalyst (i.e., a catalyst which is substantially unreactive toward fully substituted olefin carbons) under conditions that favor the removal of ethylene may afford a product triene, 2,5-dimethyl-1,3,5-hexatriene in this example. The product triene may then optionally be separated or purified, and then selectively converted to p-xylene (or a mixture of xylene isomers) via dehydrocyclization as previously described in Example 1. Separation of a product mixture comprising p-xylene, other isomeric xylenes, unreacted isoprene and/or side products may be performed to afford p-xylene (or other reaction products) at a desired purity level (e.g., >90%, >95%, >99%, etc.).

### EXAMPLE 5

### Formation of Mixed Xylenes Via Metathesis of C₅ Alkenes Over Active Metathesis Catalyst.

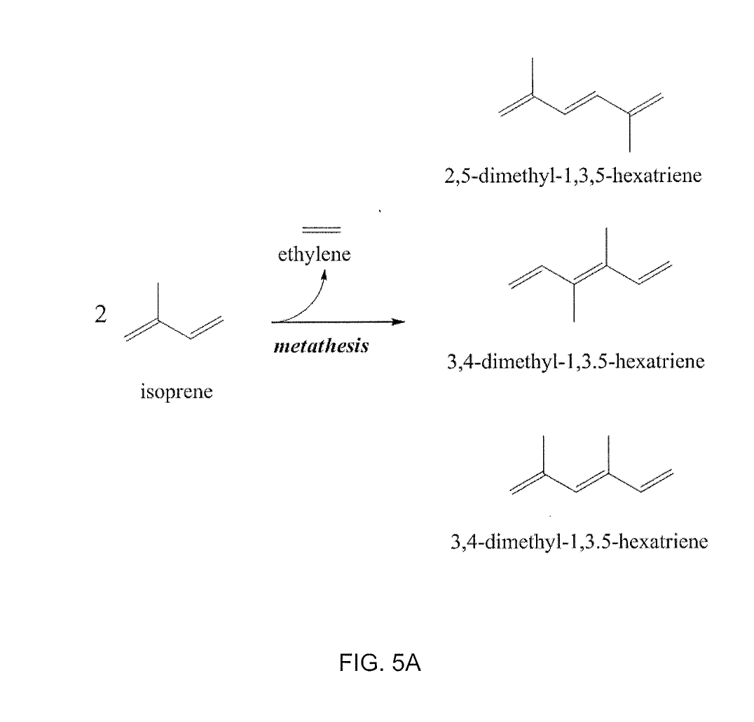

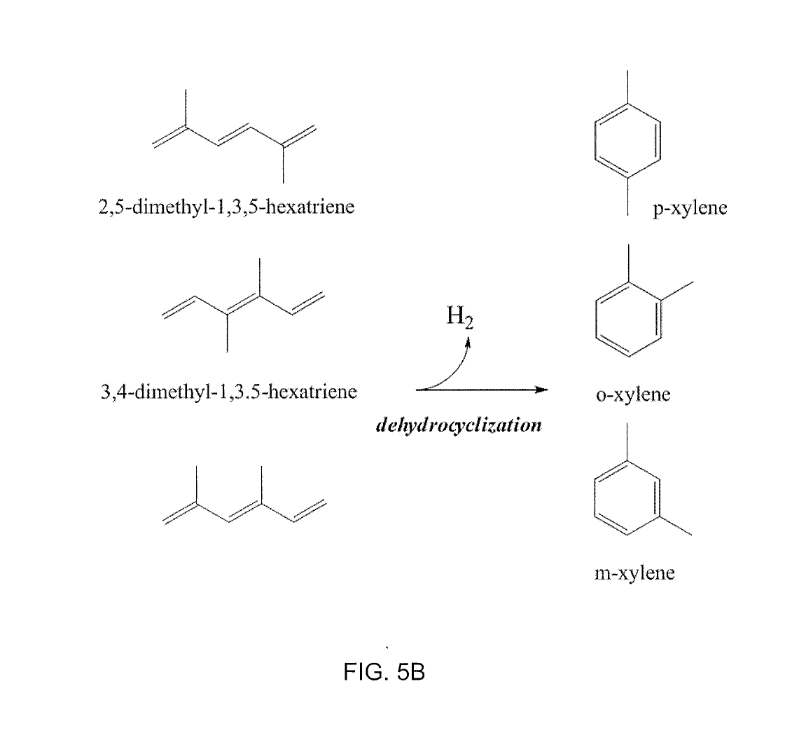

FIGS. 5A-B illustrates another embodiment of the use of diolefin, and particularly isoprene (an exemplary diolefin/C₅ alkene), in the formation of xylenes. As shown in FIG. 5, isoprene may be homometathesized over a metathesis catalyst which is active towards all olefins (e.g., is reactive toward substituted or unsubstituted olefins) under conditions that favor the removal of ethylene to form a mixture of dimethyl-1,3,5-hexatriene isomers. The resultant isomeric mixture may then be separated if desired (by methods known in the art, e.g., distillation), or the entire product stream may be subjected to further chemistry without separation. Subjecting either an isolated dimethyl hexatriene or a mixture of dimethyl-1,3,5-hexatriene isomers to dehydrocyclization conditions as previously described in Example 1 thus affords xylenes (e.g., o-xylene, m-xylene, or p-xylene, or mixtures thereof). In the case where a mixture of xylene isomers is formed, or where a product may contain undesired components such as unreacted starting material or reaction byproducts, the resultant product may be purified to provide the desired product xylene (or other reaction products) at a desired purity level (e.g., >90%, >95%, >99%, etc.). The ethylene obtained from the metathesis reaction may optionally be converted to renewable polyethylene glycol or ethylene glycol.

### EXAMPLE 6

Formation of o-Xylene Via Dehydration of C₅ Alcohols to C₅ Alkenes.

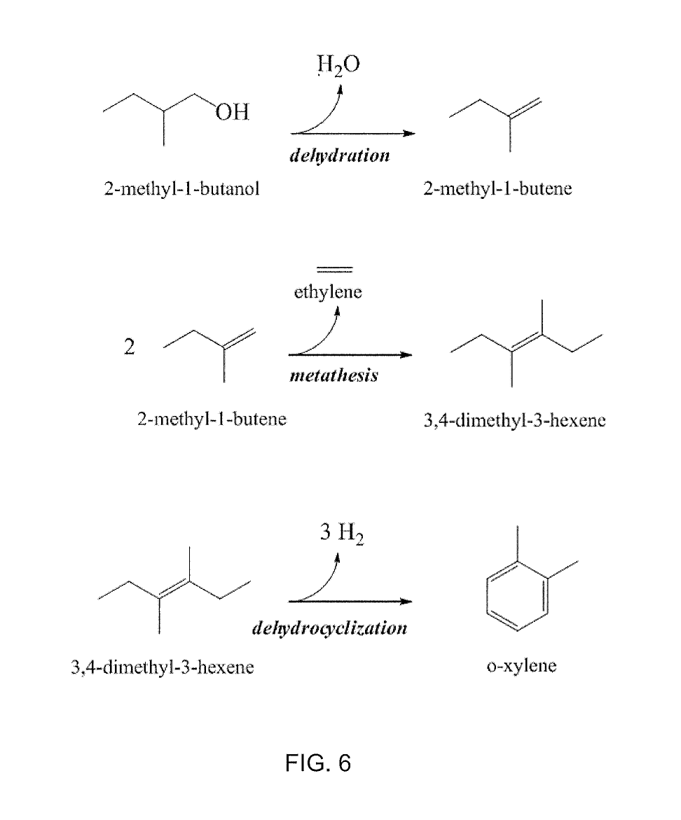

Referring to the exemplary process illustrated in FIG. 6, 2-methyl-1-butanol can be dehydrated as previously described herein to form 2-methyl-1-butene. The product 2-methyl-1-butene may then be purified if desired, or may be subject without further purification to homometathesis over a metathesis catalyst under conditions that favor removal of ethylene to form 3,4-dimethyl-3-hexene. The product 3,4-dimethyl-3-hexene may optionally be separated or purified, then selectively converted to o-xylene via dehydrocyclization as previously described in Example 1. Where a mixture of xylene isomers is formed, or where the product (in this case, o-xylene) may contain undesired components such as unreacted starting material or reaction byproducts, the resultant product may be purified to provide the desired product xylene (or other reaction products) at a desired purity level (e.g., >90%, >95%, >99%, etc.).

The processes of the present invention provide renewable xylenes, which is environmentally advantageous compared to conventional processes for preparing xylene from petrochemical feedstock. In addition, the processes of the present invention are highly selective in forming xylenes such as p-xylene, whereas conventional petrochemical processes for preparing p-xylene are relatively nonselective overall and provide a mixture of aromatic compounds, from which the p-xylene must be isolated and purified to a level suitable for e.g., production of terephthalic acid. In addition, conventional petrochemical processes for preparing p-xylene often include unit operations for separating p-xylene from by-products such as benzene, toluene, ethylbenzene, and/or for converting such by-products to xylenes (including p-xylene), and/or for isomerizing o- and m-xylenes to p-xylene. In contrast, in various embodiments of the present invention can directly provide p-xylene of sufficient purity that such purification, conversion, and isomerization steps are generally not required. That is, in most embodiments, the processes of the present invention do not include steps of separating p-xylene from other xylene isomers, or separating p-xylene from other aromatic by-products (such as those described herein), or isomerizing by-product C₈ aromatics to p-xylene. In other embodiments, only minimal purification of the p-xylene is required (e.g., by separating the p-xylene from other xylene isomers or aromatic by-products).

What is claimed is:

1. A process for preparing renewable p-xylene, comprising:
(a) treating biomass to form a feedstock;
(b) fermenting the feedstock with one or more species of microorganism, thereby forming one or more renewable C₄ or C₅ molecules, or a mixture thereof;
(c) reacting the renewable C₄ or C₅ molecules to form one or more renewable 2,5-dimethyl substituted-C₆ olefins;
(d) dehydrogenating and aromatizing at least a portion of the one or more renewable 2,5-dimethyl substituted-C₆ olefins in the presence of a dehydrocyclization catalyst to form a mixture of comprising p-xylene and hydrogen; and
(e) optionally isolating the renewable p-xylene.

2. The process of claim 1, wherein said renewable C₄ or C₅ molecules comprise renewable isobutanol, and step (c) comprises oxidizing the isobutanol to form renewable isobutyraldehyde, then condensing said renewable isobutyraldehyde with a C₄ reagent to form renewable 2,5-dimethyl-3-hexene.

3. The process of claim 1, wherein said renewable C₄ or C₅ molecules comprise a C₅ alcohol, and step (c) comprises dehydrating the C₅ alcohol to form renewable 3-methyl-1-butene, then contacting the 3-methyl-1-butene with a metathesis catalyst to form renewable 2,5-dimethyl-3-hexene.

4. The process of claim 3, wherein the C₅ alcohol is 3-methyl-1-butanol.

5. The process of claim 3, wherein said metathesis is carried out under conditions whereby ethylene is removed, thereby providing purified renewable 2,5-dimethyl-3-hexene.

6. The method of claim 5, wherein the purity of the renewable 2,5-dimethyl-3-hexene is at least 50%.

7. The process of claim 3, wherein said dehydrating is carried out in the presence of a dehydration catalyst.

8. The process of claim 1, wherein said renewable C₄ or C₅ molecules comprise a diolefin, and step (c) comprises carrying out metathesis of the diolefin to form renewable 2,5-dimethyl-1,3,5-hexatriene.

9. The process of claim 8, wherein the diolefin is isoprene.

10. The process of claim 8, wherein said metathesis is carried out under conditions whereby ethylene is removed, thereby providing purified renewable 2,5-dimethyl-1,3,5-hexatriene.

11. The method of claim 10, wherein the purity of the renewable 2,5-dimethyl-1,3,5-hexatriene is at least 50%.

12. The process of claim 8, wherein said metathesis is carried out in the presence of a metathesis catalyst.

13. The process of claim 1, wherein the one or more renewable 2,5-dimethyl substituted-C₆ olefins comprise 2,5-dimethyl-1,3,5-hexatriene.

14. The process of claim 1, wherein the one or more renewable 2,5-dimethyl substituted-C₆ olefins comprise 2,5-dimethyl-3-hexene.

15. The process of claim 1, wherein the one or more renewable 2,5-dimethyl substituted-C₆ olefins comprise 2,5-dimethyl-2,4-hexadiene.

16. The process of claim 1, wherein the renewable C₅ molecule comprises a renewable C₅ alcohol, and said reacting in step (c) comprises dehydrating the renewable C₅ alcohol to form renewable pentene, then dehydrogenating the renewable pentene to form renewable isoprene, then contacting the renewable isoprene with a metathesis catalyst to form renewable 2,5-dimethyl-1,3,5-hexatriene.

17. The process of claim 1, wherein said dehydrogenating and aromatizing of step (d) are carried out in a single reaction zone.

18. The process of claim 1, wherein said dehydrogenating and aromatizing of step (d) are carried out in two or more reaction zones.

19. The process of claim 1, wherein said dehydrocyclization catalyst is selected from the group consisting of alumina-based catalysts; silica-based catalysts; bismuth oxides; lead oxides; antimony oxides; chromium treated alumina; rhenium treated alumina; platinum treated zeolites; a mixture of chromia-alumina and bismuth oxides; bismuth oxides, lead oxides or antimony oxides in combination with supported platinum, supported palladium, supported cobalt, or metal oxides or mixtures thereof; supported chromium on a refractory inorganic oxide; rhenium oxide or metallic rhenium deposited on a neutral or weakly acidic support; platinum deposited on aluminosilicate MFI zeolite; and combinations thereof.

20. The process of claim 1, further comprising purifying the renewable 2,5-dimethyl substituted-C₆ olefins prior to the dehydrogenating and aromatizing of step (d).

21. The process of claim 1, wherein the p-xylene is isolated.

22. The process of claim 21, wherein the isolated p-xylene has a purity of greater than about 90%.
